(19) United States
(12) Patent Application Publication
(10) Pub. No.: US 2018/0344170 A1
(43) Pub. Date: Dec. 6, 2018

(54) HEART RATE DETERMINATION IN POWER-CONSTRAINED ENVIRONMENT

(71) Applicant: QUALCOMM Incorporated, San Diego, CA (US)

(72) Inventors: Robert Ganton, San Diego, CA (US); Robert Ballam, Eatons Hill (AU)

(21) Appl. No.: 15/608,774

(22) Filed: May 30, 2017

(51) Int. Cl. A61B 5/0205; A61B 5/00; G06F 17/14; G06F 17/16

(52) U.S. Cl. CPC A61B 5/0205; G06F 17/16; G06F 17/142; A61B 5/0002

(57) ABSTRACT

Methods, systems, computer-readable media, and apparatuses for power-constrained heartbeat rate determination using a device physically coupled to a user are disclosed.

### BACKGROUND

Aspects of the disclosure relate to health devices configured to determine a heart rate of a user. Health devices can be used to determine various physiological attributes of a user, such as heart rate, breathing rate, walking steps, etc. Health devices can be worn on a wrist of a user, worn on a necklace, or directly attached to a user. The health device can contain one or more sensors and a power source. Existing health devices can be incapable of determining physiological attributes of a user over extended periods of time and/or be prohibitively expensive or cumbersome. As such, there is need for improvement in the functionality of health devices.

### BRIEF SUMMARY

Certain embodiments are described that provide techniques for determining a heartbeat rate of a user in a power constrained environment (such as a wearable health device). The techniques can include use of a housing configured to be physically coupled to a user, a sensor coupled to the housing, and a controller coupled to the sensor. The controller can be configured to obtain a first characteristic vector that indicates a direction corresponding to movement induced by a heartbeat of the user. The controller can also be configured to sample, from the sensor, while the housing is physically coupled to the user, a first dataset indicative of a movement of the sensor. The controller can also be configured to generate a second dataset based on the first characteristic vector and the first dataset. The controller can determine a heartbeat rate of the user based on a frequency domain analysis of the second dataset.

The controller can be further configured to obtain a second characteristic vector that indicates a direction corresponding to movement induced by breathing of the user. The controller can also be configured to generate a third dataset based on the second characteristic vector and the first dataset. The controller can be configured to determine a breathing rate of the user based on the third dataset.

The third dataset can include the second dataset. The breathing rate of the user may not be determined based on a frequency domain analysis. The first characteristic vector and the second characteristic vector can both be obtained from one dataset acquired during a calibration cycle, wherein the dataset can be compared to respective expected ranges of movements based on a comparison to a gravity vector, each of the expected ranges of movements corresponding respectively to a heartbeat of a user and a breathing rate of the user.

The generating the second dataset can include performing a dot product multiplication of the first characteristic vector with the first dataset. The sensor can include at least one of a multi-axis accelerometer, multi-axis gyroscope, multi-axis magnetometer, multi-axis inertial measurement unit, or any combination thereof The housing can include an adhesive configured to physically couple the housing to the user. The housing can be configured to hermetically seal the controller from an external environment.

The first characteristic vector can be obtained during a calibration cycle wherein one or more detected movements are compared to an expected range of movements based on a comparison of the one or more detected movements to a gravity vector. The controller can be configured to filter to the sample dataset to reduce frequency components outside of an expected frequency range of the heartbeat rate of the user. The frequency domain analysis of the second dataset can include performing a Fast Fourier Transform on a frequency range expected to include the heartbeat rate of the user. The controller can be configured to perform the frequency domain analysis of the second dataset.

### BRIEF DESCRIPTION OF THE DRAWINGS

Aspects of the disclosure are illustrated by way of example. In the accompanying figures, like reference numbers indicate similar elements.

FIG. 1 illustrates a simplified diagram of a system that may incorporate one or more embodiments including a health device coupled to a user to determine a heartbeat rate of the user;

FIG. 2 illustrates a simplified diagram to illustrate a calibration operation and other features of the disclosure;

FIG. 3 illustrates a simplified diagram to illustrate a runtime operation and other features of the disclosure;

FIG. 4 illustrates a simplified chart to illustrate frequency domain analysis and other features of the disclosure;

FIG. 5 illustrates an example flowchart to implement calibration related features of the disclosure;

FIG. 6 illustrates an example flowchart to implement runtime related features of the disclosure; and

FIG. 7 illustrates an example of a computing system in which one or more embodiments may be implemented.

### DETAILED DESCRIPTION

Several illustrative embodiments will now be described with respect to the accompanying drawings, which form a part hereof. While particular embodiments, in which one or more aspects of the disclosure may be implemented, are described below, other embodiments may be used and various modifications may be made without departing from the scope of the disclosure or the spirit of the appended claims.

Disclosed are techniques for implementing device(s) to determine a heart rate of a user. The techniques can utilize a patch or similar health device that can be worn by a user. The patch can be a self-contained/sealed and/or disposable device that can be adhered to a user's skin (e.g., to the chest of a user). The patch can include a housing containing one or more sensors (such as an accelerometer) to determine movements or other phenomena and infer physiological attributes of a user (e.g., heartbeat rate, breathing rate, walking steps, etc.). Using such a health device, a heartbeat rate can be relatively difficult to determine due to, for example, the minimum amount of movement that may be induced in the health device as a result of beating of a heart.

The health device can also include a power source to power the one or more sensors. The health device may also include a controller and/or a transceiver to transmit sensor or other data to an external device. In certain embodiments, the disclosed health devices can be implemented via a patch that may be adhered to the body of a user. As such, it is desirable for the health device to be relatively compact and light weight to ensure that the device remains adhered to the user for a useful period of time and is unobtrusive to the user.

It is also desirable for the health device to operate for extended periods of time in order to maximize usefulness of the device (to avoid frequent removal and/or replacement of the health device). The health device may contain a power source to power the device for its intended operational lifespan (in certain embodiments, the device may be disposable). In order to minimize weight, dimensions, and cost of a health device while maximizing operational life of the device, it is desirable to minimize power usage required to determine physiological attributes of a user via the health device.

Techniques of the disclosure can be used to implemented a health device in a power-constrained environment (e.g., the aforementioned “patch”-like embodiments) in order to minimize electrical power usage requirements to power the device. Minimizing the electric power usage requirements can result in minimization of electrical power storage requirements of a power source needed to power the device for a specified amount of time. Minimizing the electrical power storage requirements can reduce a size and/or cost of the power source. Furthermore, by minimizing power usage requirements, a device can operate with less risk to a user (e.g., less risk of excessive heat generation, thermal runaway, etc.).

The disclosed techniques include various methodologies of low-power processing techniques to extract physiological attribute information from sensor information. The techniques can include determining a characteristic vector corresponding to each of a respective physiological attribute. For example, a health device may be a patch that is adhered to a chest of a user. Movement of the patch in a first vector can be indicative of a heartbeat of a user. Movement of the patch in a second vector can be indicative of a breathing rate of the same user. Because the patch is secured at a relatively stable position and orientation as compared to the user, the first and second vectors can be static.

Determining the first vector and the second vector can occur during a calibration cycle through use of a disclosed health device. During calibration, a gravity vector and/or anticipated placement of the health device in reference to a user can be used as a reference to orient the health device. After the calibration cycle is complete, the health device can store the static vector(s) that were determined. Thereafter, the vector(s) can be applied to future sensor readings in order to enhance sensor readings that correspond to the vectors (e.g., maximize components that are likely to be contributed by movement induced by a physiological attribute that is intended to be measured and/or minimize components that are not likely to be contributed by other movements).

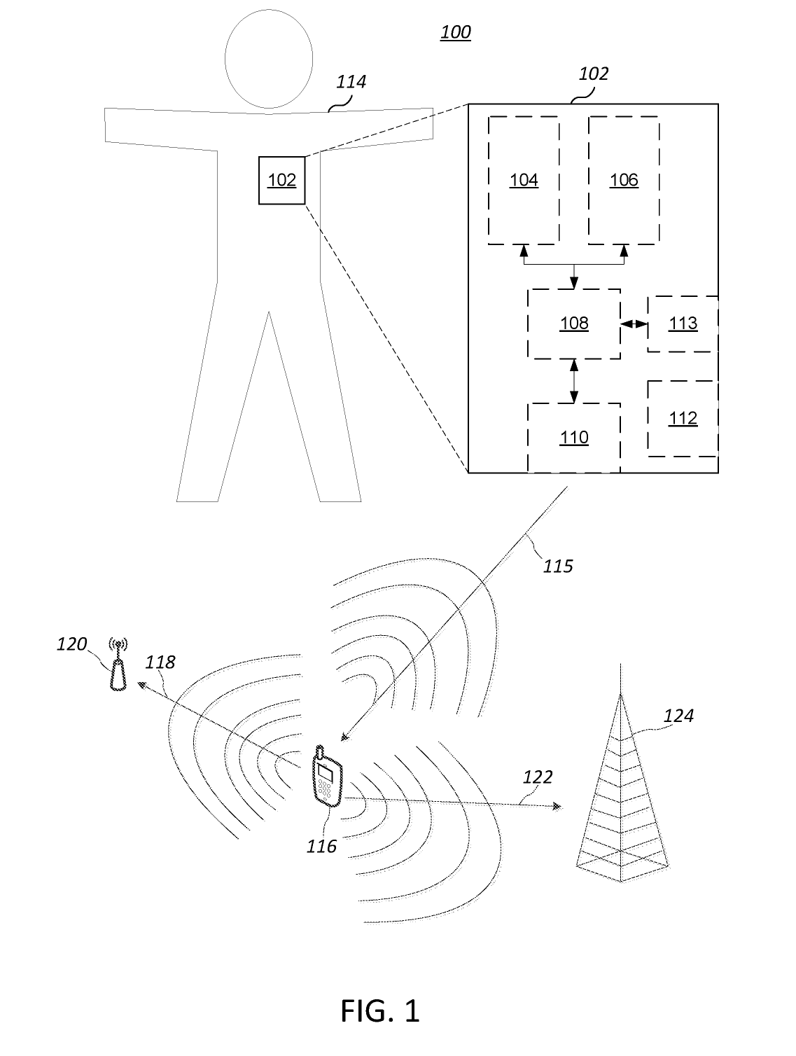

FIG. 1 illustrates a simplified diagram of a system 100 that may incorporate one or more embodiments including a health device coupled to a user to determine a heartbeat rate of the user. Illustrated is a health device 102, that can be worn as a patch (via an adhesive), on a necklace, on a belt, etc. in close proximity to a user 114. Device 102 can include an accelerometer 104 or similar sensor operable to detect movement of user 114. Accelerometer 104 can be coupled to controller 108, which can include a processor for example. Controller 108 can be configured to selectively operate in a high power state and a low power state. As disclosed herein, while in the lower power, controller 108 certain functionalities of controller 108 can be disabled in order to reduce power consumption of controller 108. While in the high power state, the functionalities of controller 108 can be enabled at the expense of requiring higher power consumption.

Sensor(s) 106 can include, without limitation sensors to determine a respiration rate, pulse rate, temperature, galvanic skin response, etc. Controller can be coupled to memory 113. Memory 113 can, for example, store one or more instructions to configure controller 108 and/or data populated by controller 108 that can indicate a level of activity of user 114. Device 102 can also include power source 112 to power components(s) of device 102 disclosed herein. Power source 112 can be a battery or capacitor, for example.

Controller 108 can be coupled to a transceiver 110 to enable wireless communication with mobile device 116. Mobile device 116 may be a device designed to perform numerous functions, including the ability to communicate, via a relatively long distance communication link, with a server (not shown). The server can collect activity information for user 114. In the example shown in FIG. 1, mobile device 116 is able to perform wireless communications by sending signals to, and receiving signals from, one or more base stations 120. For instance, mobile device 116 may send a communication signal 118 to an access point 120, which may be a base station supporting Wi-Fi communications. Mobile device 116 may send a communication signal 122 to cell tower 124, which may be a base station supporting cellular communications.

Communication signal 115 between device 102 and mobile device 116 can be relatively lower in power and/or range as compared to communication signals 118 and/or 122. In certain embodiments, device 102 may be constrained with regards to cost, operating time requirement(s), weight, and/or size and therefore relatively high power consumption components and/or batteries necessary to support longer range communications may not be integrated into device 102.

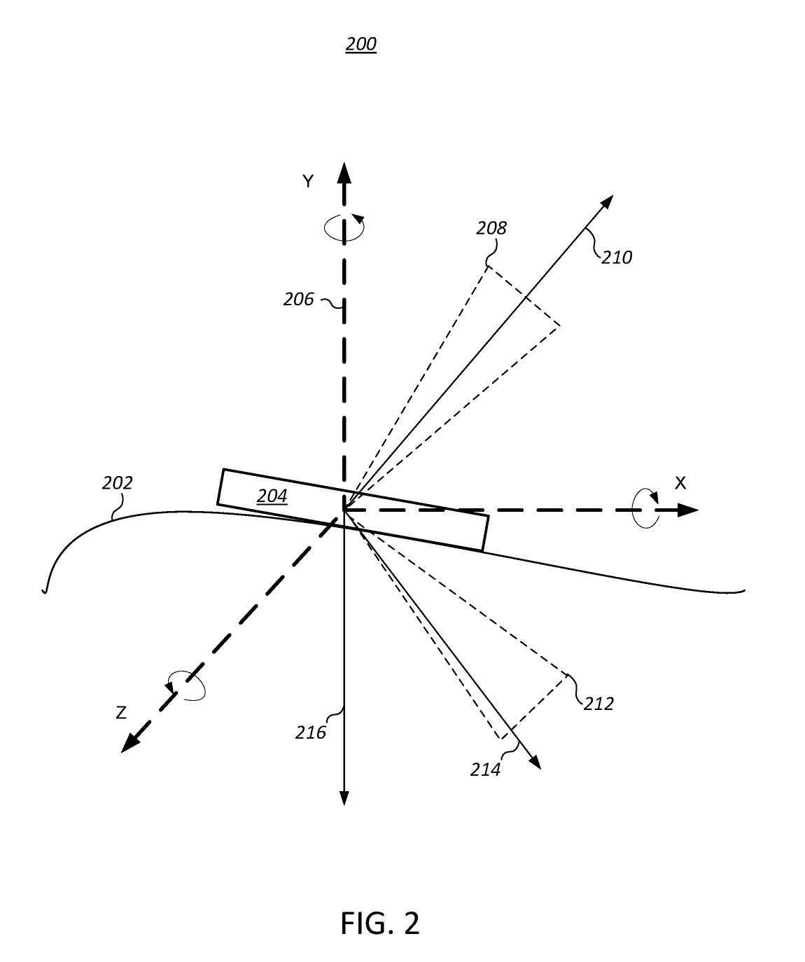

FIG. 2 illustrates a simplified diagram to illustrate a calibration mode and other features of the disclosure. System 200 can include a health device 204, which can be similar to health device 102. As illustrated, health device 204 can be attached (e.g., adhered) to skin 202 of a user (such as user 114). As disclosed herein, health device 204 can include one or more sensors, such as an accelerometer. An accelerometer can be used to determine one or more acceleration forces incident upon health device 204. Thus, when device 204 is coupled (e.g., adhered) to skin 202 (or other portions) of a user, device 204 may move or otherwise have forces incident upon device 204 in response to physiological functions of the user. For example, as a user breaths or the user's heart beats, their chest may move in response to their lungs filling with air or the muscles of their heart constricting. Device 204 may move as the chest of the user moves.

Also illustrated is a coordinate system 206 that can be used to characterize force(s) incident upon device 204. A force can be represented as a vector. As illustrated, coordinate system 206 can include three dimensions each corresponding to a corresponding dimension of three-dimensional space. Each dimension can correspond to a translational force component. Furthermore, the force can be represented with one or more rotational components (as illustrated by the arrows rotating around each of the axes of coordinate system 206). Thus, a vector, as used herein, can include one or more magnitudes and components each for a respective one of each dimensions of three-dimensional space (translational component) and/or components each for a respective one of a rotational direction around one of each dimensions of three-dimensional space.

Illustrated are three separate vectors 210, 214, and 216 that can each represent a respective force incident upon device 204. For example, vector 216 may represent a vector induced by gravity from the Earth. Thus, vector 216 can be used as a reference to determine an orientation of device 204.

Health devices disclosed herein can operate in a calibration cycle as well as a run-time cycle. The calibration cycle can be used to determine a characteristic vector for each of physiological attributes that are to be determined through use of the health device. For example, a characteristic vector can correspond to a heartbeat rate of a user. However, it should be understood that variations can exist between health devices when implemented on users due to, for example, variations in health device placement or user physiologies. To account for such variations, the aforementioned calibration cycle can be used to determine a specific characteristic vector corresponding to a physiological attribute of a specific user for which a health device is calibrated for.

A calibration cycle can include a controller of device 204 obtaining, from a user to which device 204 is coupled, a set of movement or force data representing forces induced to device 204 or movement of device 204 in response to physiological functions of the user. For example, a user may be prompted, via mobile device 116 for example, to position device 204 at a specified location and/or orientation on skin 202 of the user. The user may also be advised to position their body in a certain orientation (e.g., laying, standing, etc.), remain still for a certain amount of time, and/or refrain from or perform physical activity, for example, to alter their heartbeat rate. In certain embodiments, device 204 can monitor movements of the device to determine when the user is relatively stationary and then perform a calibration cycle. A calibration cycle can then be initiated wherein sensor(s) of device 204 may gather force or movement information over a certain amount of time. The resulting data gathered during the certain amount of time can then be used to determine one or more characteristic vectors.

As disclosed herein, a controller of device 204 may operate selectively in a low power or high power state (as may other components of device 204). In certain embodiments, device 204 may gather a single data set including movements/forces corresponding to multiple physiological attributes and use the same data set to determine multiple characteristic vectors in order to minimize power used to determine the characteristic vectors. Determining the characteristic vectors can include limiting the data set to locate vectors within corresponding ranges. For example, a range 208 is illustrated wherein a vector corresponding to a heartbeat rate of a user may located. Vector 210 may represent the actual heartbeat rate vector of the user. During calibration, movement or force vector data falling within range 208 may be analyzed and a substantially contributing/primary vector determined within the range. Thus, prior to calibration, a range 208 may be utilized to locate a vector corresponding to a heartbeat rate of a user as device 204 moves, but post calibration vector 210 may be used to locate the vector instead of range 208 (minimizing controller operations and power usage).

Similarly, range 212 can correspond to breathing of a user and similarly vector 214 may be a vector in response to a movement of device 204 induce by breathing of a particular user. During calibration, a user may be instructed to perform various actions to alter their physiological attributes (e.g., heartbeat rate, breathing rate, etc.), orientation of their body, etc. to determine several corresponding data sets/characteristic vectors that may be averaged or otherwise combined to form a singular characteristic vector for each corresponding physiological attribute.

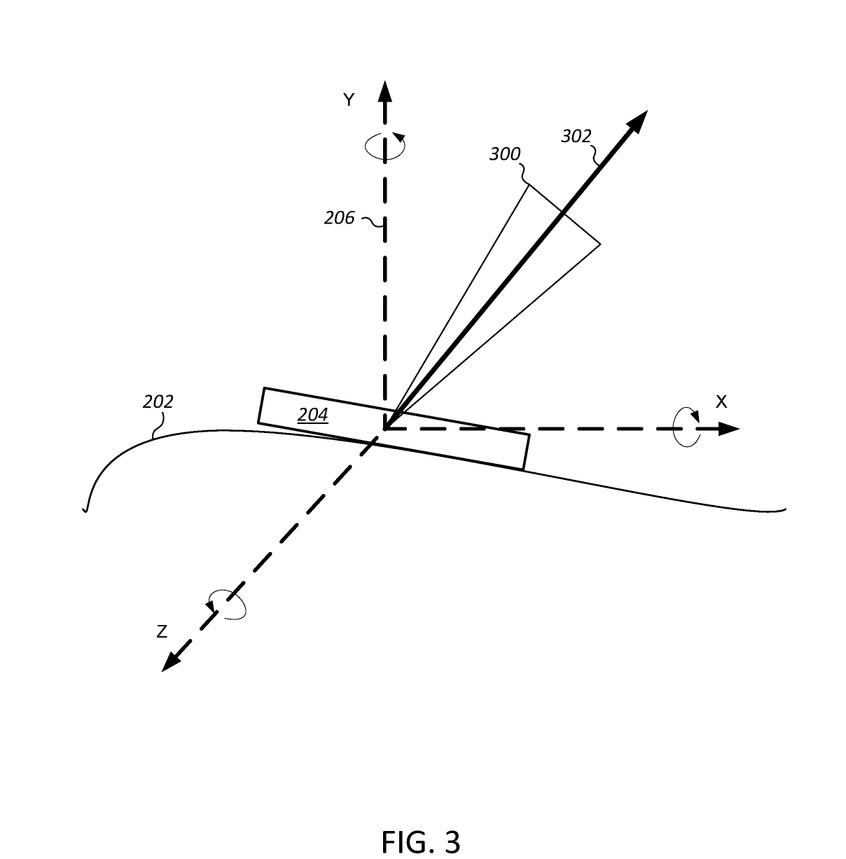

FIG. 3 illustrates a simplified diagram to illustrate a runtime operation and other features of the disclosure. Illustrated is device 204 post calibration cycle (e.g., in runtime operation). As illustrated, characteristic vector 302 is not being generated by a controller of device 204 during the runtime operation. Instead, characteristic vector 302 was previously determined during the calibration operation. The characteristic vector may correspond to vector 210, for example, or may be an average/combination of multiple vectors. During runtime, instead of searching or attempting to identify vectors that may correspond to movement of device 204, device 204 may instead enhance received movement data using vector 302. For example, a movement data set obtained at run time may be dot product multiplied by vector 302. By dot product multiplying vector 302 with movement data, components of the movement data in the direction of vector 302 can be enhanced and remaining components diminished. For example, any movement data falling within range 300 may become more pronounced and therefore may be easier to identify.

By making the movement within range 300 easier to identify, a controller may have less difficult ascertaining a movement induced by a specific physiological attribute corresponding to a characteristic vector used to enhance the movement data. For example, a controller may require less processing to remove noise or other movements within the movement data. Thus, the controller may require less electrical power to characterize the physiological attribute as compared to techniques not utilizing the characteristic vector. Furthermore, dot product multiplication can be performed with relatively little electrical power and low processor overhead. In addition, multiple types of movement may be extracted/enhanced in this manner using one set of movement data. For example, heartbeat rate movement can be extracted/enhanced by dot product multiplication with one characteristic vector (e.g., vector 210), and breathing movement can be extracted/enhanced by dot product multiplication with another characteristic vector (e.g., vector 214), all using the same set of movement data. Often, operation of sensors (e.g., an accelerometer) to gather movement data represents a significant portion of the power consumption of a health device, especially for a low-power device. By extracting multiple types of movements/physiological attributes from the same set of movement data, the health device can reduce the amount of power it consumes. The same movement data can be used for multiple purposes.

Further post-processing may be performed on the movement data after being enhanced by characteristic vector 302. For example, frequency domain or other analyses may be performed on the data to further characterize a physiological attribute. This may be especially true for determination of a heartbeat rate of a user due to the minimal movement that may be induced in a device as a result of a heartbeat. The movement induced by the heartbeat may be difficult to differentiate from noise (e.g., other movements or sensor noise). Thus, frequency domain analysis may aid in differentiating movement components cause by a heartbeat rate and other movements. Frequency domain analysis can be relatively power intensive which may be undesirable for a health device disclosed herein. Techniques are disclosed to minimize the power utilized by a frequency domain analysis.

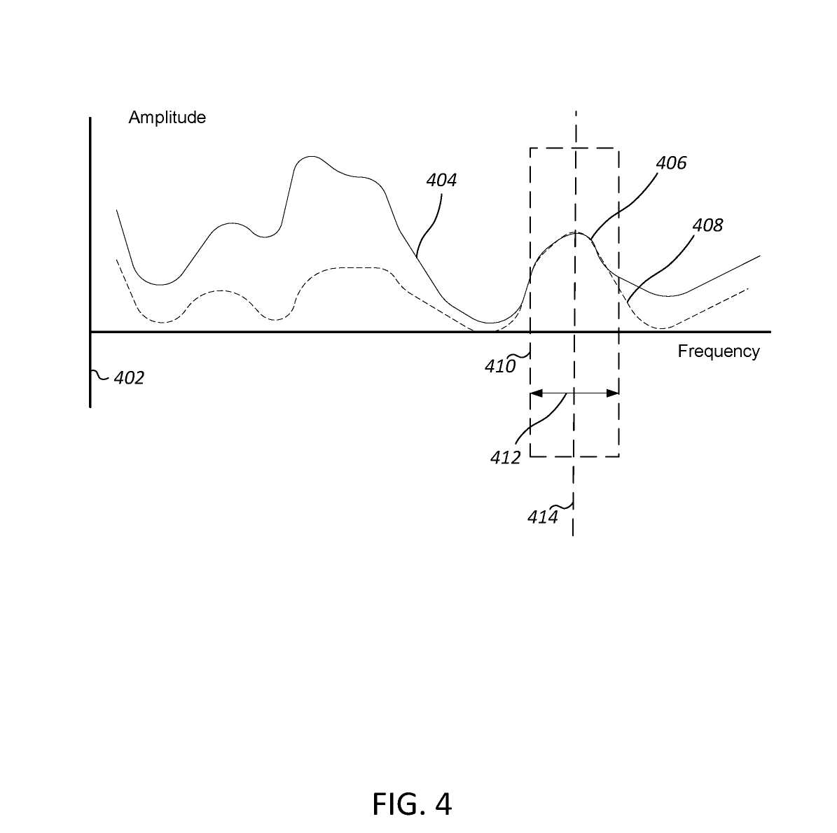

FIG. 4 illustrates a simplified chart to illustrate frequency domain analysis and other features of the disclosure. Illustrated is a frequency domain representation of a signal both pre and post enhancement. Illustrated is a graph 402 with amplitude on the Y axis and frequency on the X axis. As illustrated, a signal captured over time can represent movement of device 204, for example. Frequency domain signal 404 can represent the signal captured over time. The frequency domain signal can be determined using Fourier Transform or other techniques. As illustrated, a certain range of frequencies 412 within a region 410 of graph 402 may correspond to a heartbeat rate of a user. For example, range of frequencies 412 may correspond to a heartbeat in the range of fifty to two hundred and fifty beats per minute. Range of frequencies 412 may be further defined during a calibration cycle for a specific individual.

Frequency 414 may be a central or expected heart rate for an individual and may be determined based on one or other sensor of a health device or a time of day, for example. For example, a user may be expected to be sleeping during certain times of day and may therefore have a relatively consistent heartbeat rate during that time. Either range of frequencies 412 or frequency 414 can be used to enhance components of signal 404 within region 410. For example, portion 406 of signal 404 may be enhanced (as illustrated by 408) within region 410 by band pass filtering within range of frequencies 412 or around frequency 414, for example. Band pass filtering can be performed on a time domain or frequency domain representation of a signal and may require minimal electrical power by a controller. In certain embodiments, signal 404 can be enhanced within region 410 by performing a Fast Fourier Transform within range of frequencies 412 or around frequency 414, for example, to obtain a frequency domain representation of signal 404 within region 410.

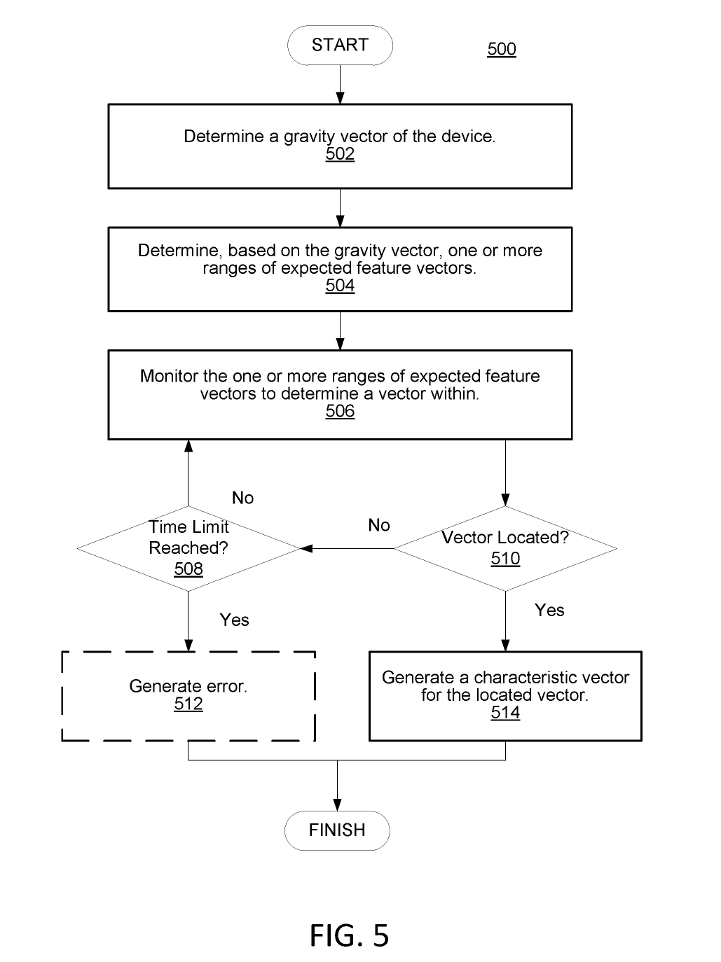

FIG. 5 illustrates a flowchart 500 to implement calibration related features of the disclosure. At 502, a gravity vector incident upon the device can be determined. For example, the gravity vector may be induced by a force of gravity induced by the Earth. At 504, one or more ranges of expected feature vectors can be determined based on the gravity vector. As disclosed, a user may be directed or expected to position a device at a certain location and/or orientation of their body. The gravity vector can be used to verify and/or adjust the orientation of the device for a specific user. For example, a device (such as a patch) may be oriented differently on a chest of a body builder as opposed to a marathon runner. Thus, the expected range of feature vectors may also be adjusted according to a specific orientation of a deice when positioned on a specific user. For example, a feature vector range can correspond to a heartbeat rate of a user and be adjusted based on the specific orientation of a device on a user.

At 506, the one or more ranges of expected feature vectors can be monitored to determine a vector within each of the one or more ranges. Each vector can correspond to movement induced by a heartbeat rate, breathing, walking, etc. As disclosed herein, the vector can be an amalgamation/combination of several vectors. At 510, a determination can be made if a vector was determined within the expected range. If not, then, at 508, a determination can be made if a time limit is reached. If the time limit has not been reached, then the process can proceed to 506 to further monitor the one or more ranges of expected features. If the time limit was reached, then a device may optionally generate an error 512 or otherwise be unable to determine a characteristic vector.

If a vector was located at 510, then a characteristic vector can be generated for the located vector. As disclosed herein, the characteristic vector can be a combination of located vectors and/or multiple characteristic vectors may be located corresponding to one dataset from readings from sensor(s) of a device over a common time period. Thus, steps 508, 510, 512, and 514 can be performed for each of the one or more ranges of 506. The characteristic vector(s) determined at 514 can be stored within memory of a health device for late retrieval during runtime.

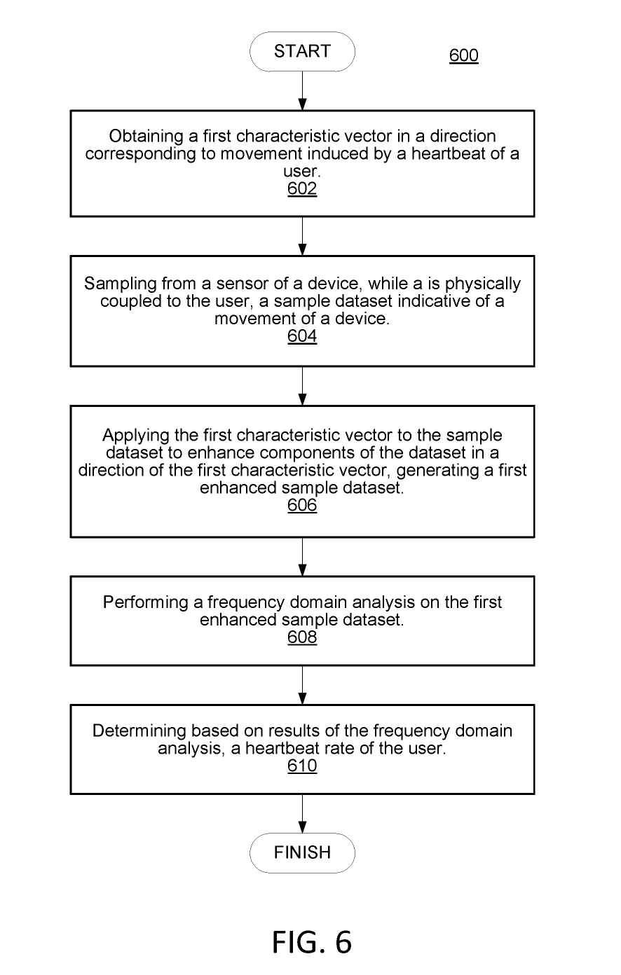

FIG. 6 illustrates a flowchart 600 for determining a heartbeat rate of a user using features of the disclosure. At 602, a first characteristic vector can be obtained. The first characteristic vector can correspond to a heartbeat rate of a user. The first characteristic vector can be determined using the process of flowchart 500 and may be stored in memory of a health device. At 604, a sensor of a device can be used to sample a dataset indicative of movement of the device while the device is physical coupled to the user. As disclosed herein, the dataset may include multiple vectors each corresponding to a physiological attribute of the user.

At 606, the first characteristic vector can be applied to the sample dataset to enhance components of the dataset in a direction of the first characteristic vector, generating a first enhanced sample dataset. Applying the first characteristic vector can include dot product multiplying the characteristic vector with the dataset. The enhanced sample dataset can include enhanced components of the sample data set in the direction of the first characteristic vector and/or reduced other components. As disclosed herein, an enhanced sample dataset can be generated for each characteristic vector/physiological attribute or for multiple characteristic vectors/physiological attributes.

At 608, a frequency domain analysis can be performed on the first enhanced sample dataset. The first enhanced sample dataset can include a time component. The frequency domain analysis can be performed to differentiate movement induced by a heartbeat of a user from other movement (e.g., noise or from other physiological functions). The frequency domain analysis can optionally include band pass limiting/filtering. At 610, a heartbeat rate of the user can be determined based on results of the frequency domain analysis. For example, an impulse or a peek can be determined based on a frequency domain representation of a sensor dataset, the frequency of the impulse or peek corresponding to the heartbeat rate of the user.

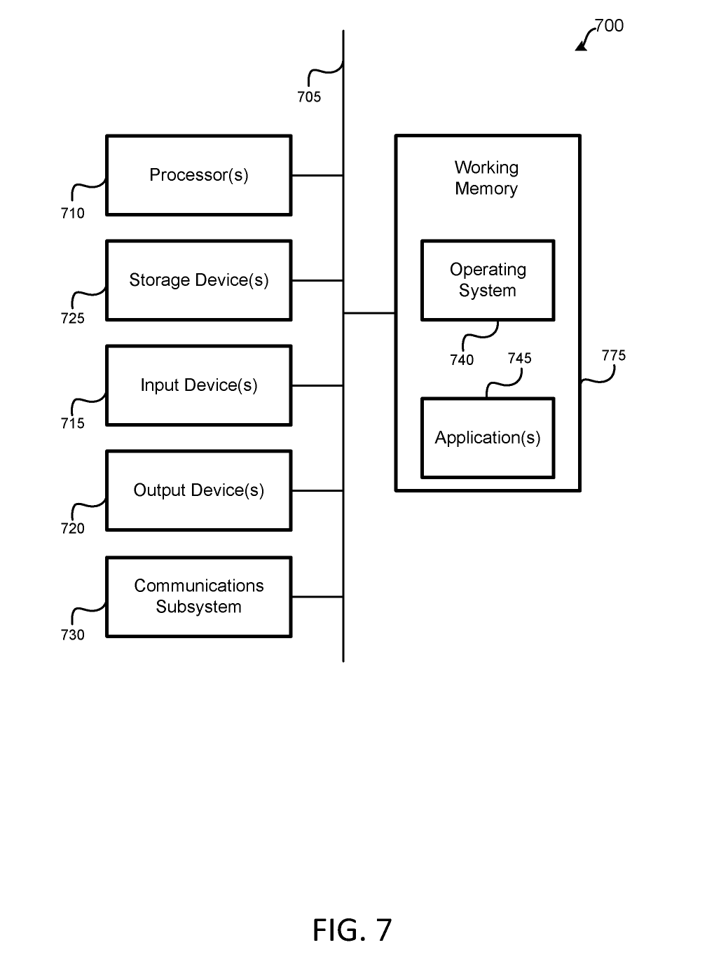

FIG. 7 illustrates an example computer system that can implement functionality of certain components, such as controller 108. The computer system 700 is shown comprising hardware elements that can be electrically coupled via a bus 705 (or may otherwise be in communication, as appropriate). The hardware elements may include one or more processors 710, including without limitation one or more general-purpose processors and/or one or more special-purpose processors (such as digital signal processing chips, graphics acceleration processors, video decoders, and/or the like); one or more input devices 715, which can include without limitation a mouse, a keyboard, remote control, and/or the like; and one or more output devices 720, which can include without limitation a display device, a printer, and/or the like. As used herein, a controller can include functionality of a processor (such as processors 710).

The computer system 700 may further include (and/or be in communication with) one or more non-transitory storage devices 725, which can comprise, without limitation, local and/or network accessible storage, and/or can include, without limitation, a disk drive, a drive array, an optical storage device, a solid-state storage device, such as a random access memory (“RAM”), and/or a read-only memory (“ROM”), which can be programmable, flash-updateable and/or the like. Such storage devices may be configured to implement any appropriate data stores, including without limitation, various file systems, database structures, and/or the like.

The computer system 700 might also include a communications subsystem 730, which can include without limitation a modem, a network card (wireless or wired), an infrared communication device, a wireless communication device, and/or a chipset (such as a Bluetooth™ device, an 802.11 device, a Wi-Fi device, a WiMax device, cellular communication device, GSM, CDMA, WCDMA, LTE, LTE-A, LTE-U, etc.), and/or the like. The communications subsystem 730 may permit data to be exchanged with a network (such as the network described below, to name one example), other computer systems, and/or any other devices described herein. In many embodiments, the computer system 700 will further comprise a working memory 775, which can include a RAM or ROM device, as described above.

The computer system 700 also can comprise software elements, shown as being currently located within the working memory 775, including an operating system 740, device drivers, executable libraries, and/or other code, such as one or more application programs 745, which may comprise computer programs provided by various embodiments, and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a non-transitory computer-readable storage medium, such as the non-transitory storage device(s) 725 described above. In some cases, the storage medium might be incorporated within a computer system, such as computer system 700. In other embodiments, the storage medium might be separate from a computer system (e.g., a removable medium, such as a compact disc), and/or provided in an installation package, such that the storage medium can be used to program, configure, and/or adapt a general purpose computer with the instructions/code stored thereon. These instructions might take the form of executable code, which is executable by the computer system 700 and/or might take the form of source and/or installable code, which, upon compilation and/or installation on the computer system 700 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer system (such as the computer system 700) to perform methods in accordance with various embodiments of the invention. According to a set of embodiments, some or all of the procedures of such methods are performed by the computer system 700 in response to processor 710 executing one or more sequences of one or more instructions (which might be incorporated into the operating system 740 and/or other code, such as an application program 745) contained in the working memory 775. Such instructions may be read into the working memory 775 from another computer-readable medium, such as one or more of the non-transitory storage device(s) 725. Merely by way of example, execution of the sequences of instructions contained in the working memory 775 might cause the processor(s) 710 to perform one or more procedures of the methods described herein.

The terms “machine-readable medium,” “computer-readable storage medium” and “computer-readable medium,” as used herein, refer to any medium that participates in providing data that causes a machine to operate in a specific fashion. These mediums may be non-transitory. In an embodiment implemented using the computer system 700, various computer-readable media might be involved in providing instructions/code to processor(s) 710 for execution and/or might be used to store and/or carry such instructions/code. In many implementations, a computer-readable medium is a physical and/or tangible storage medium. Such a medium may take the form of a non-volatile media or volatile media. Non-volatile media include, for example, optical and/or magnetic disks, such as the non-transitory storage device(s) 725. Volatile media include, without limitation, dynamic memory, such as the working memory 775.

Common forms of physical and/or tangible computer-readable media include, for example, a floppy disk, a flexible disk, hard disk, magnetic tape, or any other magnetic medium, a CD-ROM, any other optical medium, any other physical medium with patterns of marks, a RAM, a PROM, EPROM, a FLASH-EPROM, any other memory chip or cartridge, or any other medium from which a computer can read instructions and/or code.

Various forms of computer-readable media may be involved in carrying one or more sequences of one or more instructions to the processor(s) 710 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 700.

The communications subsystem 730 (and/or components thereof) generally will receive signals, and the bus 705 then might carry the signals (and/or the data, instructions, etc. carried by the signals) to the working memory 775, from which the processor(s) 710 retrieves and executes the instructions. The instructions received by the working memory 775 may optionally be stored on a non-transitory storage device 725 either before or after execution by the processor(s) 710.

It should further be understood that the components of computer system 700 can be distributed across a network. For example, some processing may be performed in one location using a first processor while other processing may be performed by another processor remote from the first processor. Other components of computer system 700 may be similarly distributed. As such, computer system 700 may be interpreted as a distributed computing system that performs processing in multiple locations. In some instances, computer system 700 may be interpreted as a single computing device, such as a distinct laptop, desktop computer, or the like, depending on the context.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

Wireless communication techniques described herein may be in connection with various wireless communications networks such as a wireless wide area network (“WWAN”), a wireless local area network (“WLAN”), a wireless personal area network (WPAN), and so on. The term “network” and “system” may be used interchangeably herein. A WWAN may be a Code Division Multiple Access (“CDMA”) network, a Time Division Multiple Access (“TDMA”) network, a Frequency Division Multiple Access (“FDMA”) network, an Orthogonal Frequency Division Multiple Access (“OFDMA”) network, a Single-Carrier Frequency Division Multiple Access (“SC-FDMA”) network, or any combination of the above networks, and so on. A CDMA network may implement one or more radio access technologies (“RATs”) such as cdma2000, Wideband-CDMA (“W-CDMA”), to name just a few radio technologies. Here, cdma2000 may include technologies implemented according to IS-95, IS-2000, and IS-856 standards. A TDMA network may implement Global System for Mobile Communications (“GSM”), Digital Advanced Mobile Phone System (“D-AMPS”), or some other RAT. GSM and W-CDMA are described in documents from a consortium named “3rd Generation Partnership Project” (“3GPP”). Cdma2000 is described in documents from a consortium named “3rd Generation Partnership Project 2” (“3GPP2”). 3GPP and 3GPP2 documents are publicly available. 4G Long Term Evolution (“LTE”) communications networks may also be implemented in accordance with claimed subject matter, in an aspect. A WLAN may comprise an IEEE 802.11x network, and a WPAN may comprise a Bluetooth network, an IEEE 802.15x, for example. Wireless communication implementations described herein may also be used in connection with any combination of WWAN, WLAN or WPAN.

In another aspect, as previously mentioned, a wireless transmitter or access point may comprise a cellular transceiver device, utilized to extend cellular telephone service into a business or home. In such an implementation, one or more mobile devices may communicate with a cellular transceiver device via a code division multiple access (“CDMA”) cellular communication protocol, for example.

Techniques described herein may be used with an SPS that includes any one of several GNSS and/or combinations of GNSS. Furthermore, such techniques may be used with positioning systems that utilize terrestrial transmitters acting as “pseudolites”, or a combination of SVs and such terrestrial transmitters. Terrestrial transmitters may, for example, include ground-based transmitters that broadcast a PN code or other ranging code (e.g., similar to a GPS or CDMA cellular signal). Such a transmitter may be assigned a unique PN code so as to permit identification by a remote receiver. Terrestrial transmitters may be useful, for example, to augment an SPS in situations where SPS signals from an orbiting SV might be unavailable, such as in tunnels, mines, buildings, urban canyons or other enclosed areas. Another implementation of pseudolites is known as radio-beacons. The term “SV”, as used herein, is intended to include terrestrial transmitters acting as pseudolites, equivalents of pseudolites, and possibly others. The terms “SPS signals” and/or “SV signals”, as used herein, is intended to include SPS-like signals from terrestrial transmitters, including terrestrial transmitters acting as pseudolites or equivalents of pseudolites.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein.

Therefore, it is intended that claimed subject matter not be limited to the particular examples disclosed, but that such claimed subject matter may also include all aspects falling within the scope of appended claims, and equivalents thereof.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

If implemented in firmware and/or software, the functions may be stored as one or more instructions or code on a computer-readable storage medium. Examples include computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM or other optical disk storage, magnetic disk storage, semiconductor storage, or other storage devices, or any other medium that can be used to store desired program code in the form of instructions or data structures and that can be accessed by a computer; disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

In addition to storage on computer-readable storage medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. That is, the communication apparatus includes transmission media with signals indicative of information to perform disclosed functions. At a first time, the transmission media included in the communication apparatus may include a first portion of the information to perform the disclosed functions, while at a second time the transmission media included in the communication apparatus may include a second portion of the information to perform the disclosed functions.

What is claimed is:

1. A device, comprising:
a sensor coupled to a housing configured to be physically coupled to a user;
a controller coupled to the sensor, the controller configured to:
obtain a first characteristic vector that indicates a direction corresponding to movement induced by a heartbeat of the user;
obtain, from the sensor, while the housing is physically coupled to the user, a first dataset indicative of a movement of the sensor;
generate a second dataset based on the first characteristic vector and the first dataset; and
determine a heartbeat rate of the user based on a frequency domain analysis of the second dataset.

2. The device of claim 1, wherein the controller is further configured to:
obtain a second characteristic vector that indicates a direction corresponding to movement induced by breathing of the user;
generate a third dataset based on the second characteristic vector and the first dataset; and
determine a breathing rate of the user based on the third dataset.

3. The device of claim 2, wherein the third dataset includes the second dataset.

4. The device of claim 2, wherein the breathing rate of the user is not determined based on a frequency domain analysis.

5. The device of claim 2, wherein the first characteristic vector and the second characteristic vector are each obtained from a dataset acquired during a calibration cycle, wherein the dataset is compared to respective expected ranges of movements based on a comparison to a gravity vector, each of the expected ranges of movements corresponding respectively to a heartbeat of a user and a breathing rate of the user.

6. The device of claim 1, wherein the generating the second dataset includes performing a dot product multiplication of the first characteristic vector with the first dataset.

7. The device of claim 1, wherein the sensor includes at least one of a multi-axis accelerometer, multi-axis gyroscope, multi-axis magnetometer, multi-axis inertial measurement unit, or any combination thereof.

8. The device of claim 1, further comprising an adhesive configured to physically couple the housing to the user.

9. The device of claim 1, wherein the housing is configured to hermetically seal the controller from an external environment.

10. The device of claim 1, wherein the first characteristic vector is obtained during a calibration cycle wherein one or more detected movements are compared to an expected range of movements based on a comparison of the one or more detected movements to a gravity vector.

11. The device of claim 1, wherein generating the second dataset includes filtering the first dataset to reduce frequency components outside of an expected frequency range of the heartbeat rate of the user.

12. The device of claim 1, wherein the frequency domain analysis of the second dataset includes performing a Fast Fourier Transform on a frequency range expected to include the heartbeat rate of the user.

13. The device of claim 1, wherein the controller is configured to perform the frequency domain analysis of the second dataset.

14. A method, comprising:
obtaining, by a controller, a first characteristic vector that indicates a direction corresponding to movement induced by a heartbeat of a user;
obtaining, by the controller, from a sensor physically coupled to a housing, while the housing is physically coupled to the user, a first dataset indicative of a movement of the sensor;
generating, by the controller, a second dataset based on the first characteristic vector and the first dataset; and
determining, by the controller a heartbeat rate of the user based on a frequency domain analysis of the second dataset.

15. The method of claim 14, further comprising:
obtaining, by the controller, a second characteristic vector that indicates a direction corresponding to movement induced by breathing of the user;
generating, by the controller, a third dataset based on the second characteristic vector and the first dataset; and
determining, by the controller a breathing rate of the user based on the third dataset.

16. The method of claim 15, wherein the third dataset includes the second dataset.

17. The method of claim 15, wherein the breathing rate of the user is not determined based on a frequency domain analysis.

18. The method of claim 15, wherein the first characteristic vector and the second characteristic vector are both obtained from one dataset acquired during a calibration cycle, wherein the dataset is compared to respective expected ranges of movements based on a comparison to a gravity vector, each of the expected ranges of movements corresponding respectively to a heartbeat of a user and a breathing rate of the user.

19. The method of claim 14, wherein the generating the second dataset includes performing a dot product multiplication of the first characteristic vector with the first dataset.

20. An apparatus, comprising:
a means for obtaining a first characteristic vector that indicates a direction corresponding to movement induced by a heartbeat of a user;
a means for obtaining, from a sensor physically coupled to a housing, while the housing is physically coupled to the user, a first dataset indicative of a movement of the means for sampling;
a means for generating a second dataset based on the first characteristic vector and the first dataset; and
a means for determining a heartbeat rate of the user based on results of a frequency domain analysis of the second dataset.

21. The apparatus of claim 20, further comprising:
a means for obtaining a second characteristic vector that indicates a direction corresponding to movement induced by breathing of the user;
a means for generating a third dataset based on the second characteristic vector and the first dataset; and
a means for determining a breathing rate of the user based on the third dataset.

22. The apparatus of claim 21, wherein the third dataset includes the second dataset.

23. The apparatus of claim 21, wherein the breathing rate of the user is not determined based on a frequency domain analysis.

24. The apparatus of claim 21, wherein the first characteristic vector and the second characteristic vector are both obtained from one dataset acquired during a calibration cycle, wherein the dataset is compared to respective expected ranges of movements based on a comparison to a gravity vector, each of the expected ranges of movements corresponding respectively to a heartbeat of a user and a breathing rate of the user.

25. The apparatus of claim 20, wherein the generating the second dataset includes performing a dot product multiplication of the first characteristic vector with the first dataset.

26. One or more computer readable medium comprising instructions that, when executed by one or more processors, cause the one or more processors to:
obtain a first characteristic vector that indicates a direction corresponding to movement induced by a heartbeat of a user;
obtain, from a sensor physically coupled to a housing, while the housing is physically coupled to the user, a first dataset indicative of a movement of the sensor;
generate a second dataset based on the first characteristic vector and the first dataset; and
determine a heartbeat rate of the user based on frequency domain analysis of the second dataset.

27. The one or more computer readable medium of claim 26, further comprising instructions that, when executed by the one or more processors, cause the one or more processors to:
obtain a second characteristic vector that indicates a direction corresponding to movement induced by breathing of the user;
generate a third dataset based on the second characteristic vector and the first dataset; and
determine a breathing rate of the user based on the third dataset.

28. The one or more computer readable medium of claim 27, wherein the third includes the second dataset.

29. The one or more computer readable medium of claim 27, wherein the breathing rate of the user is not determined based on a frequency domain analysis.

30. The one or more computer readable medium of claim 27, wherein the first characteristic vector and the second characteristic vector are both obtained from one dataset acquired during a calibration cycle, wherein the dataset is compared to respective expected ranges of movements based on a comparison to a gravity vector, each of the expected ranges of movements corresponding respectively to a heartbeat of a user and a breathing rate of the user.
